(19) United States
(12) Patent Application Publication
(10) Pub. No.: US 2014/0005469 A1
(43) Pub. Date: Jan. 2, 2014

(54) IMPLANTS FOR STRESS URINARY INCONTINENCE TREATMENTS AND RELATED METHODS

(71) Applicant: Urova Medical, Inc., Memphis, TN (US)

(72) Inventors: Franklin Yao, New York, NY (US); Adam Xiao, Fallston, MD (US); Kelsey Humphries, Boise, ID (US); Shida Li, West Chester, PA (US); Michael Chen, Portland, OR (US); Amanda Ojeda, Easton, PA (US); Logan Howard, Surfside Beach, SC (US)

(21) Appl. No.: 13/923,874

(22) Filed: Jun. 21, 2013

Related U.S. Application Data
(60) Provisional application No. 61/665,047, filed on Jun. 27, 2012.

(51) Int. Cl. A61F 2/00

(52) U.S. Cl. CPC A61F 2/0036
USPC 600/30; 29/592

(57) ABSTRACT

An urogynecologic implant has a curved body that disperses force and reduces the ability of the urethra to expand into the pelvic floor under impulses of abdominal pressure in order to inhibit, reduce or prevent stress urinary incontinence.

### RELATED APPLICATIONS

This application claims the benefit of and priority to U.S. Provisional Application Ser. No. 61/665,047, filed Jun. 27, 2012, the contents of which are hereby incorporated by reference as if recited in full herein.

### FIELD OF THE INVENTION

The present invention relates to surgical implants to treat urinary incontinence.

### BACKGROUND

Stress incontinence is the involuntary leakage of urine due to increased abdominal pressure such as during a cough. This is particularly prevalent in women and has been shown to degrade the quality of life. In the past, transvaginal slings, transobturator tape, and a single-incision mini-slings have been used to attempt to treat this condition. Generally stated, these devices include a surgical mesh that is placed around the mid-urethra to form a hammock-like structure, anchoring it to the pubic bone to attempt to prevent the leakage of urine.

However, the transvaginal and transobturator slings require three incisions and the surgery is performed in a hospital operating room, making it inconvenient and expensive. The mini-sling only requires a single incision, but it has been associated with a lower effectiveness and a higher risk of complications, which have recently come under FDA review.

### SUMMARY OF EMBODIMENTS OF THE INVENTION

Embodiments of the present invention provide minimally invasive surgical implants that can inhibit or prevent stress urinary incontinence.

The implants can be placed and/or implanted to operate in a non-restrictive way to increase the ease of implantation and prevent voiding complications.

Embodiments of the invention are directed to stress incontinence implants. The implants have a rigid or semi-rigid urogynecologic implant comprising a first primary surface with a curved medial portion and a second primary surface underlying the first primary surface.

The implant can have a monolithic porous polymer body.

The implant can have s a hollow interior compartment.

The implant can be sized and configured for transvaginal placement.

The implant can have a width dimension and a length dimension and the width dimension can be greater than the length dimension.

The width dimensions can be about 50% greater than the length dimension.

The second primary surface can be curved and can have a radius of curvature that is greater than a radius of curvature of the first primary surface medial portion.

The first primary surface can have a medial portion that merges into outer ramped end portions. The second primary surface can have an arc configuration with opposing ends and a respective ramped end portion can meet a respective arc end portion to define a respective short end edge of the implant.

The short end edges can reside closer to the first primary surface than the second primary surface.

The implant can be configured to reside between an anterior vagina wall and an outer surface of a lower to middle portion of a urethra.

The implant can be held in position without mechanical fixation between the vagina wall and the urethra.

The first primary surface medial portion can merge into outer ramped end portions that incline down toward second primary surface. The implant can include mesh extending out from the respective ramped end portions.

The implant can be sized and configured to surround only about 180 degrees or less of a female urethra and can have a maximum thickness that is between about 1 mm to about 5 mm.

The implant can have a width that is between about 15-30 mm and a length that is between about 7-10 mm and a maximum thickness that is between about 1 mm and 5 mm.

The implant cam be a silicone body.

The implant can have a porosity of at least one of: (i) a matrix of pores, (b) spaced apart or intersecting channels; or (c) pores and channels, at least some of which have a diameter between about 125 mm and 250 mm.

The implant can have a biodegradeable body.

The implant can be provided in a plurality of different sizes according to severity of stress urinary incontinence and/or urethra size, wherein at least one of implant width or implant thickness increases for implants for severe stress urinary incontinence and large urethra size, relative to implants for mild or moderate stress urinary incontinence and small or medium urethra size.

Other embodiments are directed to methods of treating urinary incontinence. The methods include: (a) placing a three-dimensional shaped rigid or semi-rigid implant between a urethra and anterior vagina wall of a female patient; then (b) distributing forces from a first side of the implant facing the urethra to a longer second side of the implant adjacent the anterior vagina wall in response to impulses of abdominal pressure stress to thereby inhibit urinary incontinence.

The placing can be carried out via entry in a single incision in the vagina wall.

Still other embodiments are directed to methods of fabricating a urinary incontinence implant. The methods include forming a rigid or semi-rigid three dimensional implant body having a three-dimensional shape including a first radius of curvature associated with a medial portion of a first primary surface and a second radius of curvature associated with an arc of an underlying second primary surface.

The method can include forming pores in the implant body during or after the molding.

The forming the pores can include directing laser light into the molded implant body to form through channels.

Before the forming step, the method can include providing a flowable material of the moldable material that can be combined with a porogen in the mold or prior to introducing into the mold, then after the forming step, the porogon can be removed from the molded implant body leaving a porous implant body.

The forming step can include injection molding implant material in a mold having a cavity that defines the first and second radii of curvature.

The implant material can include silicone.

Still other embodiments are directed to molds for a medical stress incontinence implant. The molds include a mold body having an internal volumetric cavity with walls that are configured with first and second radii of curvature that are configured to form a rigid or semi-rigid three dimensional implant body having a three-dimensional shape including a first radius of curvature associated with a medial portion of a first primary surface and a second radius of curvature associated with an arc of an underlying second primary surface.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

Other systems and/or methods according to embodiments of the invention will be or become apparent to one with skill in the art upon review of the following drawings and detailed description. It is intended that all such additional systems, methods, and/or devices be included within this description, be within the scope of the present invention, and be protected by the accompanying claims.

### BRIEF DESCRIPTION OF THE DRAWINGS

Other features of the present invention will be more readily understood from the following detailed description of exemplary embodiments thereof when read in conjunction with the accompanying drawings.

FIG. 1 is a top perspective view of one example of a surgical implant according to embodiments of the present invention.

FIG. 2 is a side view along one long edge of the implant shown in FIG. 1.

FIG. 3 is a side view along one short edge of the implant shown in FIG. 1.

FIG. 4 is a top view of the implant shown in FIG. 1.

FIG. 5 is a top perspective view illustrating an exemplary implant with mesh attachments according to some embodiments of the present invention.

FIGS. 6A-6C are schematic illustrations of a response of the pubourethral system to a cough without the present invention.

FIGS. 7A-7C are schematic illustrations of the response of the pubourethral system to a cough with the present invention according to embodiments of the present invention.

FIG. 8 is an enlarged schematic illustration of an implant in position according to embodiments of the present invention.

FIGS. 9A-9C are schematic illustrations of a sequence of surgical steps that can be used to place urogynecologic implants according to embodiments of the present invention.

FIGS. 10A-10C are illustrations of exemplary dimensions and radii according to particular embodiments of the present invention.

FIG. 11 is a side section view (taken inward of one long edge) of the implant shown in FIG. 1 illustrating a hollow interior cavity according to embodiments of the present invention.

FIG. 12 is a side view (along one long edge) of an exemplary implant illustrating different end configurations and that the implant may include one or more pores or apertures according to embodiments of the present invention.

FIG. 13 is a perspective view of an implant similar to that shown in FIG. 1 illustrating that the implant can have sufficient pores to allow fluid transport therethrough according to embodiments of the present invention.

FIG. 14 is a schematic illustration of different size implants selected to accommodate different size urethras according to embodiments of the present invention.

FIG. 15 is a schematic illustration of different size implants selected to accommodate different degrees of severity of urinary stress incontinence according to embodiments of the present invention.

FIG. 16A is a side perspective view of an exemplary mold for fabricating an implant according to embodiments of the present invention.

FIG. 16B is a section view of the mold shown in FIG. 16A according to embodiments of the present invention.

FIG. 17 is a schematic illustration of a method/system for forming pores in a stress incontinence implant according to some embodiments of the present invention.

FIG. 18 is a schematic illustration of another embodiment of a method/system for forming pores in a stress incontinence implant according to some embodiments of the present invention.

### DETAILED DESCRIPTION OF EMBODIMENTS OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise. One or more features shown and discussed with respect to one embodiment may be included in another embodiment even if not explicitly described or shown with another embodiment.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “about” means that the recited number or value can vary by +/−20%.

The term “sterile” means that the noted device or material meets or exceeds defined medical guidelines of cleanliness and is substantially (if not totally) without contaminants so as to be suitable for medical uses.

The term “urogynecologic implant” refers to implants targeted for use between a urethra and vagina of female patients. The implants may be acutely or chronically placed. The implants may be for medical or veterinarian uses, e.g., for human or animals, but are particularly suitable for human use.

The term “semi-rigid” means that the implant is flexible but has sufficient rigidity to substantially maintain its three-dimensional shape under normal loading in the body.

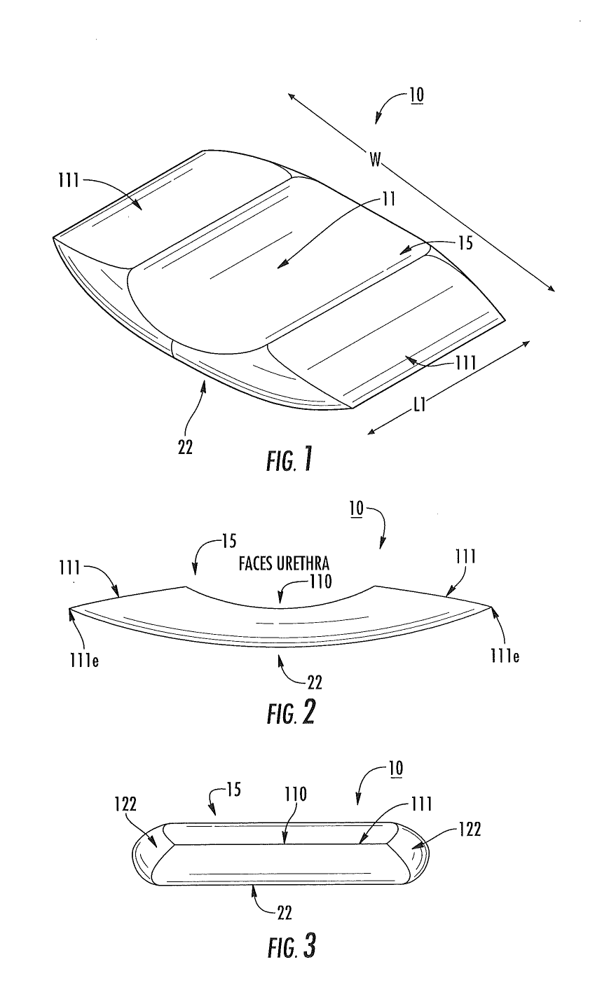

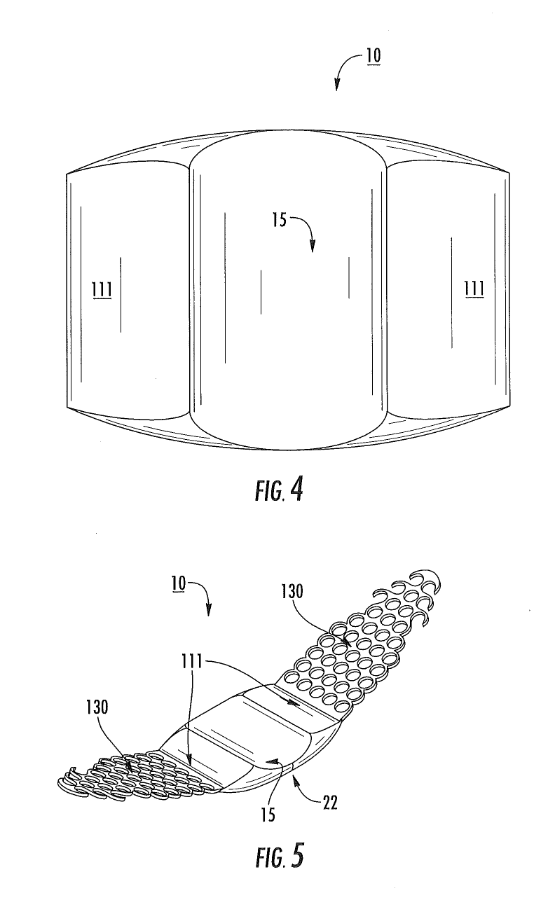

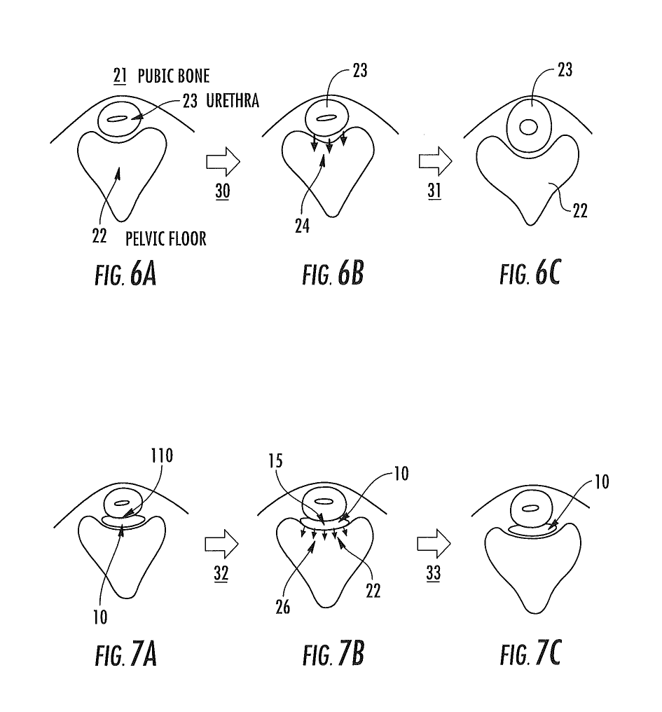

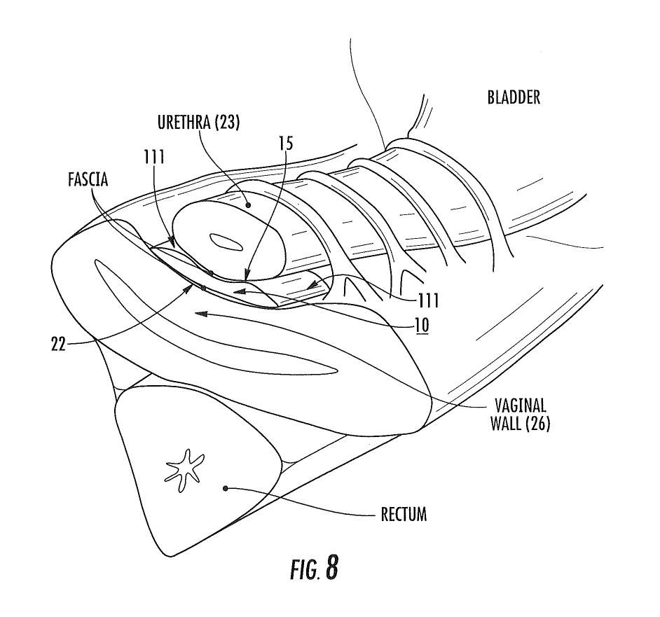

Referring now to FIGS. 1-4, there is shown an implant 10 having a rigid or semi-rigid curved shape. The implant 10 has two opposing primary surfaces 15, 22. The first primary surface 15 is curved in a direction facing a urethra 23 (FIG. 7A, 8). In the orientation shown, the first primary surface 15 is a top surface. The first primary surface 15 can include a medial portion that is curved or has a relatively wide groove 110 that merges into two downwardly-curved or linearly tapered end portions 110 that can terminate at an outermost edge 110e (which may be sharp, blunt or rounded). The other primary surface 22 can also be upwardly curved (or curved in a direction facing the urethra 23) and meet the upper primary surface 15 at the edge 111e. The second primary surface 22 may also be substantially planar (not shown).

The implant 10 can have a width “W” that is about the size of a diameter of an average urethra (e.g., about 20 mm). The length dimension “L” is typically less than the W dimension, typically between about 40-60% less, such as about 50% less. The length L1 (at the ends) can be about ⅓ the length of an average lower end length of a urethra, e.g., about 10 mm. The maximum length dimension L2 may be at the center and be greater than the length L1 (at the ends 111 as shown in FIG. 10C and this may be about 14 mm.

The maximum thickness (average) can be between about 1 mm to about 10 mm, typically 1 mm to 9 mm, including between about 1-6 mm, such as about 1.5 mm, about 2 mm, about 2.5 mm, about 3 mm, about 3.5 mm, about 4 mm, about 4.5 mm or about 5 mm.

The surface area of the curved surface or groove 110 can be about 50% of the longer surface 22 as shown in FIGS. 1 and 10C. However, this relationship can vary and it is contemplated that a less incontinent woman may use an implant where the groove or curved surface 110 is closer in area to the longer second primary surface 22 while a more incontinent woman may use an implant where the shorter curved or groove surface 110 has an area that is even less than about 50% than the longer surface to provide increased support.

In some embodiments, the implant 10 can be sized and configured to surround only about 180 degrees or less of a female urethra, typically between about 60-120 degrees, and can have a maximum thickness that is between about 1 mm to about 6 mm, typically about 1.5 mm to about 5 mm, and in some embodiments about 2 mm, about 2.5 mm, about 3 mm, about 3.5 mm, about 4 mm, about 4.5 mm or about 5 mm.

In some embodiments, the implant 10 can have a width W that is between about 15-30 mm, a length L that is between about 7-10 mm and a maximum thickness that is between about 1 mm and about 5 mm.

FIGS. 10A-10C illustrate exemplary radii and dimensions (and reflect average dimensions) of the implant 10. As shown, the implant 10 can include three different radii, a first radius R1 defining the curvature of the groove or middle portion 110 of the upper surface 15, a second radius R2 defining the curvature of the lower or second primary surface 22 and an optional third radius R3 for defining the interface between the surfaces 15, 22. The first radius R1 can have a dimension that is about 10 mm measured from a centerline (marked as “C/L” as shown in FIG. 10A). The second radius R2 can be at least about double the first, such as about 27 mm. The third radius R3, where used, can be the largest radius, typically about 30-50% greater than R2, and may be about 39.4 mm. The edge 111e may reside closer to the first primary surface 15 than the second 22, e.g., at about 1.8 mm on a side having an overall thickness of about 2 mm.

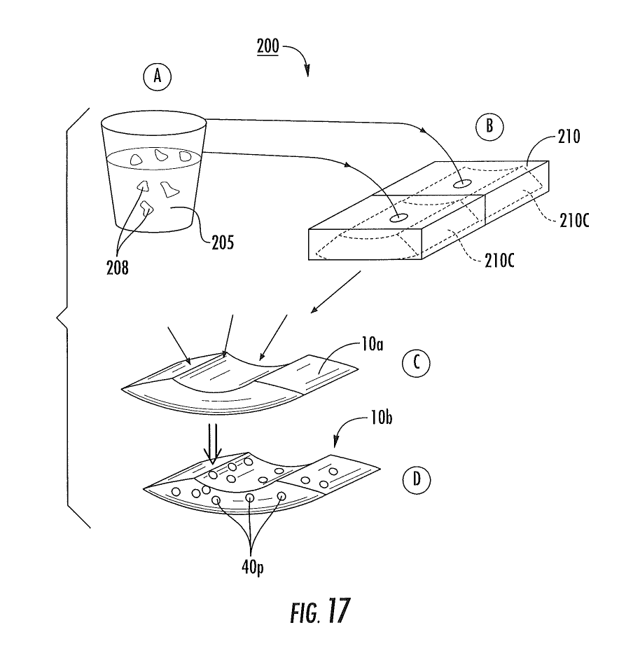

The shapes and size of the implant 10 can vary from that shown. Also, as shown in FIGS. 14 and 15, the implant 10 can be provided in a range of different sizes for different anatomical and/or functional requirements or fit. For example, the implants 10 can be provided in a plurality of defined ranges, such as “small,” “medium” and “large” with different lengths, widths and/or potentially thicknesses and/or differing degrees of rigidity. The middle portion 110 of the surface 15 may change as the ratio R of a shorter to longer surface 15:22 changes. Also, the thickness of the implant and overall shape (and rigidity) may vary according to other factors such as the degree of pelvic prolapse.

FIG. 14 illustrates that the maximum width W (the width of the longest side dimension) is typically 10-25% greater than the outer diameter of the lower to mid portion of the urethra, at the location the implant 10 is to be positioned. As shown, for a small urethra, e.g., about a 1.4 cm diameter urethra, the longest length of a suitable implant can be about 1.6 cm or about 1.7 cm; for a medium size urethra, e.g., about a 1.7 cm diameter urethra, the maximum outer length of the implant can be about 2.0 cm; and for a large urethra, e.g., about a 2.0 cm diameter urethra, a 2.4 cm implant may be appropriate. The thickness dimension (measured at the centerline) of the implant 10 can vary, including between about 2.5 mm to about 6 mm, typically between about 3 mm to about 5 mm, on average, measured at the centerline C/L.

FIG. 15 illustrates that the size/configuration of an implant 10 can be selected according to the severity of the stress urinary incontinence, e.g., mild (Mi), moderate (Mo) or severe Sv, with the width dimension W being wider in that order, such as from about 1. 6 cm, to about 2.0 cm, to about 2.4 cm or other increasing widths. Again, the thickness and/or rigidity of the implant may also vary according to a rated severity of the condition.

The curvatures of primary surfaces 15 and 22 can be shaped to fit about adjacent surrounding anatomy. In position, the first primary surface 15 can contact an outer surface of a lower to mid-portion of the urethra 23 (FIG. 7A, 8) while the bottom or lower primary surface 22 rests on top of an outer surface of the (anterior) vaginal wall 26, as shown in FIGS. 7A and 8.

The primary surfaces 15 and 22 can be completely smooth to reduce stress on tissue and inhibit complications such as erosion or extrusion. The term “smooth” means that there is a smooth (rather than rough) tactile feel so that its surface finish is non-irritating to adjacent tissue. The primary surfaces 15 and 22 can be continuous solid closed outer surfaces, and, at least for the portions contacting local tissue, may have a constant continuous and uninterrupted line (radius).

In some embodiments, the surface area of the curved middle portion or groove 110 of the first primary surface 15 can be smaller than the surface area of the rear or second primary surface 22. The radius of curvature of surfaces of the outer segments 111 can be greater than that of both 110 and 22 in order to create a disparity between the contact area of the groove (110) and urethra 23, and the contact area of the bottom surface (22) and vaginal wall 26. The two primary surfaces 15, 22 can meet at edges 111e and at outer rounded ends or shoulders 122 (FIG. 3).

In some embodiments, the implant 10 can be a rigid or semi-rigid molded body. The implant 10 can be formed of a biocompatible (inert) material or materials such as a biocompatible polymer(s) or rubber that provides sufficient rigidity to be able to provide the force distribution. In some embodiments, the implant 10 can have a Young's modulus between about 2 and about 10 MPa, substantially corresponding to the range of elasticity between healthy and weakened vaginal wall tissue.

The implant 10 can have a porous body (FIG. 13) or a solid hollow body with a hollow interior 20 (FIG. 11) or combinations of same. The implant 10 can be a monolithic molded body of a biocompatible (non-cytotoxic) material that has a defined three-dimensional shape. In some embodiments, the implant 10 can be a solid, porous and/or hollow monolithic molded body of a plurality of biocompatible materials that define the three-dimensional shape. In some embodiments, the implant 10 comprises polypropylene. In some embodiments, the implant 10 comprises silicone. In some embodiments, the implant 10 is rigid or semi-rigid and comprises a combination of materials and may be formed of a material(s) that is/are radio-opaque or biodegradeable over time, such as, but not limited to, polycaprolactone and poly-L-lactone.

The implant 10 may be coated, impregnated, painted, sprayed, dipped or otherwise formed to include (externally and/or internally) a radio-opaque material, such as barium sulfate, to allow in-vivo imaging. The implant 10 may be coated, impregnated or otherwise formed with a biocompatible (non-cytotoxic) material, such as collagen, to reduce the risk of infection. The implant 10 can incorporate therapeutic agents or drugs that are released to local tissue over time. The term “drug” is used interchangeably with “therapeutic agent” and refers to an agent (e.g., an organic compound, an inorganic compound, a biomolecule, etc.) that has a beneficial effect on a subject/patient, which beneficial effect can be complete or partial. “Biomolecule” as used herein refers to a protein, a polypeptide, a nucleic acid (e.g., a deoxyribonucleic acid and/or a ribonucleic acid), and/or a fragment thereof. Exemplary drugs include, but are not limited to, analgesics such as non-steroidal anti-inflammatory drugs and opioids; antibiotics; anti-scarring agents; steroids; anti-inflammatory agents such as steroids, salicylates, ibuprofen, naproxen, dexamethasone, prednisolone, corticosterone, budesonide, estrogen, sulfasalazine and mesalamine; bisphosphonates; anti-thrombotic agents such as heparin, heparin derivatives, urokinase, and PPack (dextrophenylalanine proline arginine chloromethylketone); antineoplastic/antiproliferative/anti-miotic agents such as paclitaxel, 5-fluorouracil, cisplatin, vinblastine, vincristine, epothilones, endostatin, angiostatin, angiopeptin, and thymidine kinase inhibitors; anesthetic agents such as lidocaine, bupivacaine and ropivacaine; vascular cell growth promoters such as transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); antimicrobial agents such as triclosan, cephalosporins, aminoglycosides and nitrofurantoin; cytotoxic agents, cytostatic agents and cell proliferation affectors; vasodilating agents; antibodies (e.g., monoclonal antibodies and/or polyclonal antibodies); growth factors; cytokines; hormones; vitamins; minerals; or any combination thereof.

Referring now to FIG. 5, a length or section of macroporous surgical mesh 13 may be optionally attached to both ends 111 of the implant 10. This may encourage tissue integration and improve device fixation over a long period of time. The mesh segments 13 can be configured from any suitable material and may optionally comprise small, interwoven threads of polypropylene. The mesh 13 can be rigid, semi-rigid or flexible. The mesh ends 13 can be tucked into surrounding tissue (rather than locked together). They can be positioned with about a 137 degree angle between them, and can extend towards the obturator membrane.

Referring now to FIGS. 6A-6C, a section of the pubourethral anatomy is shown. This anatomy includes a pubic bone 21, urethra 23, and pelvic floor 22 which is comprised of the vaginal wall 26, rectum, and levator ani. FIGS. 6A-6C display the response of a stress incontinent pubourethral system without the invention to a sudden impulse of abdominal pressure 30. Generally stated, the pubic bone 21 is fixed in place, but the increase in intra-urethral force causes the urethra 23 to exert a downward force 24 on the pelvic floor 22. The result 31 can be that the urethra 23 expands and sinks into the pelvic floor 22, thus allowing leakage to occur.

In contrast, as shown in FIGS. 7A-7C, the implant 10, when positioned in the pubourethral anatomy, can inhibit urine leakage. The surface contact area between the urethra 23 and medial portion or groove 110 is smaller than the contact area between the pelvic floor and lower or second primary surface 22. Thus, during an impulse of abdominal pressure 32, the downward force 26 of the urethra is dispersed (distributed) across a greater area and the associated pressure is reduced. The result 33 is that the pelvic floor becomes better able to resist the urethral expansion and inhibit, reduce or prevent leakage. The implant 10 may also optionally be configured to act as a shock absorber to be able to reduce the degree of force transmitted.

FIG. 8 illustrates the implant 10 in position. The implant 10 can be “free-floating” or self-restraining to be held in position once placed. This is because the holding space that the implant is placed an artificial sub-urethral space due to the surgery (this area is typically filled with fascia and connective tissue). Thus, it is unlikely that the implant 10 will migrate. These two tissue compartments can hold the implant 10 in position without requiring positive fixation. Optionally, sutures or surgical glue may be used to attach the device to the pubourethral fascia to facilitate fixation. Over time, the implant 10 may be encapsulated by fibrous tissue or in-growth which can connect or attach it to the urethra and/or vaginal wall.

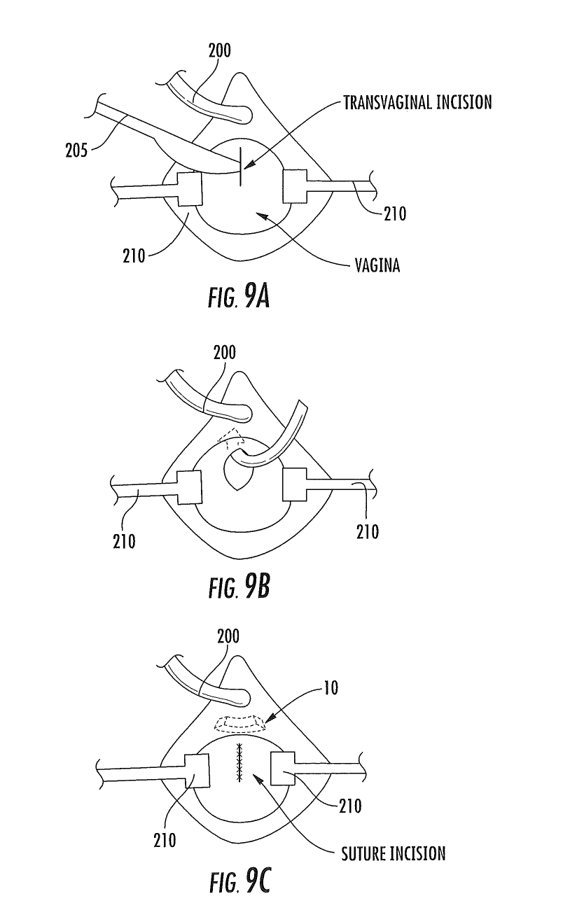

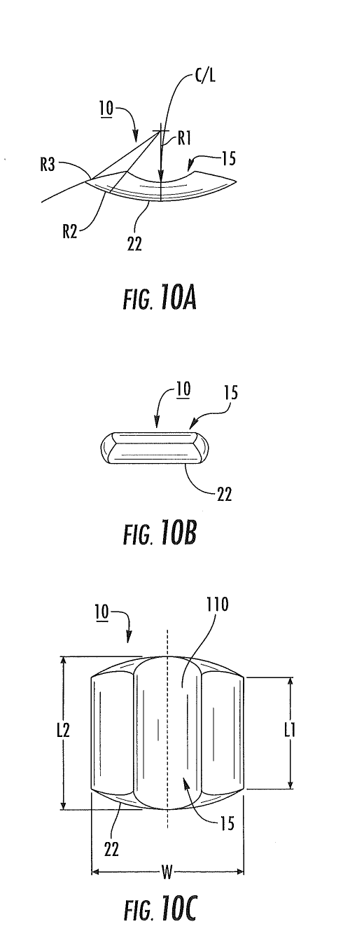

FIGS. 9A-9C illustrate an exemplary sequence of steps for placing the implant 10, typically with a single, transvaginal incision. A urethral catheter 200 can be used with clamps 210 to hold the vagina open. The urethra can be palpated with a finger, then the insert can be inserted into the appropriate space between the urethra and vagina wall as shown in FIG. 8. If the implant 10 with mesh ends 13 is placed, then a trochar may be used to place or position the mesh. Ultrasound or another imaging modality may be used to facilitate proper placement or confirm placement of the implant 10.

FIG. 11 illustrates another embodiment of an implant 10. In this embodiment the implant 10 has a hollow interior compartment 20. This compartment 20 may include a different material such as a resilient material or the compartment 20 can include air (non-pressurized). In some embodiments, an internal pressurized bladder may reside in the interior compartment and may comprise saline or other inert fluid. In some embodiments, the implant 10 includes an outer casing that does not require a bladder (it may be sufficiently air or fluid tight) and can have a pressurized compartment of air, saline or other material.

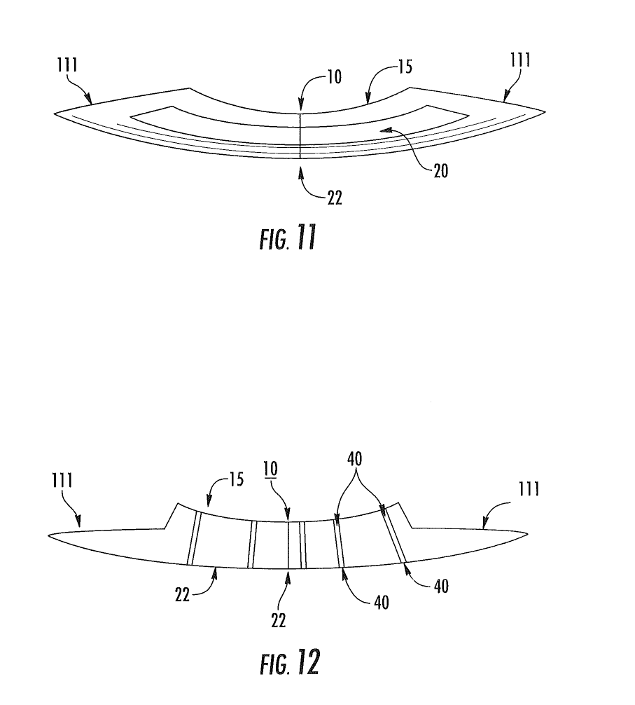

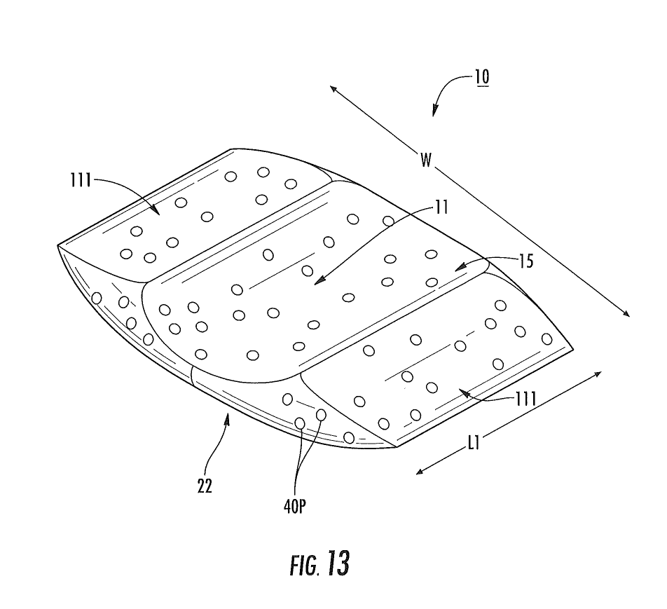

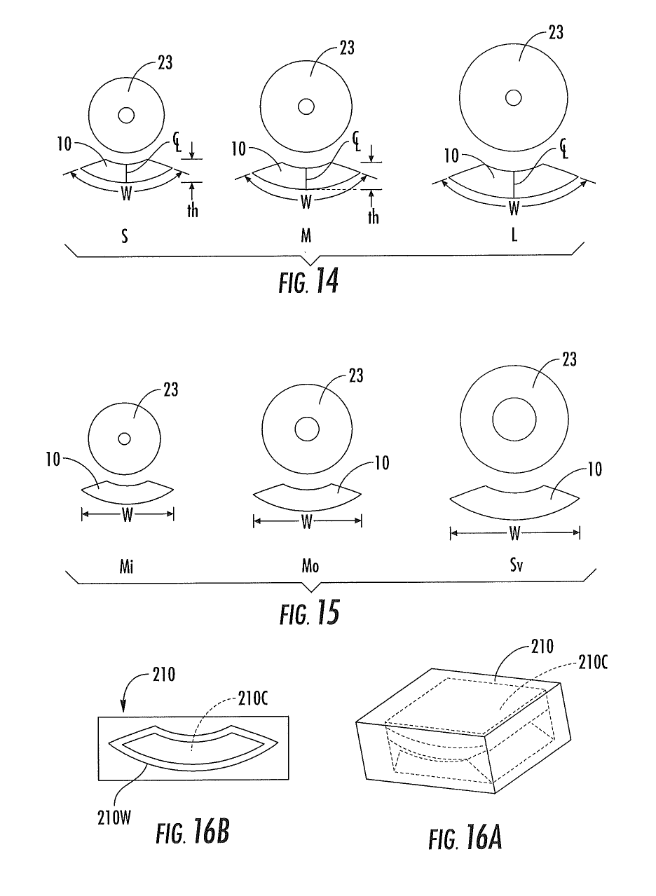

FIG. 12 illustrates that the implant 10 can include a different profile shape on the upper or first primary surface 10.

FIG. 12 also illustrates that the implant 10 can include a plurality of spaced apart apertures 40 (shown as through apertures, but closed ones may also be used). The apertures 40 may allow fluid transfer and/or tissue ingrowth. The apertures 40 can extend in a common direction, top to bottom and/or side to side, and can be parallel or may intersect or be discrete spaced apart channels.

FIG. 13 shows the implant can include pores 40p that may be in a regular or irregular pattern and that may be discrete or interconnected as a matrix to provide channels for fluid flow or exchange through the implant 10. The pores 40p and/or apertures 40a can be sized and configured to allow for white blood cell migration and/or tissue integration. The pores 40p and/or apertures 40 can be configured to provide between about 10-60% porosity, typically between about 10-40% porosity, with aperture and/or pore diameters ranging between about 10 mm to about 300 mm, such as between about 125 mm and 250 mm, including about 125 mm, about 130 mm, about 140 mm, about 150 mm, about 160 mm, about 170 mm, about 180 mm, about 190 mm, about 200 mm, about 210 mm, about 220 mm, about 230 mm, about 240 mm and about 250 mm.

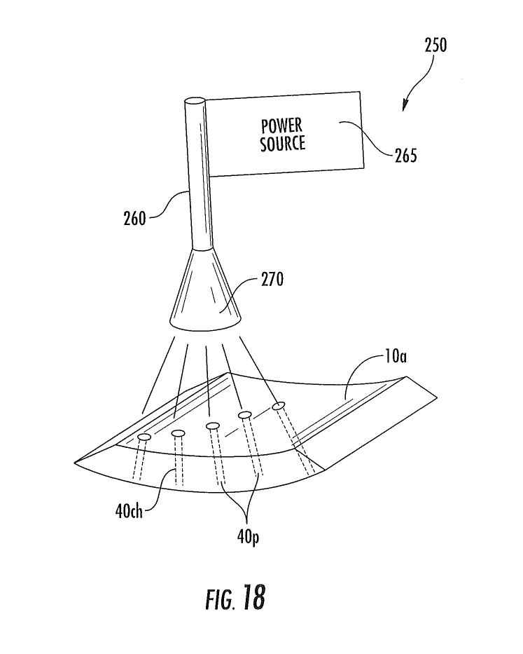

FIGS. 16A and 16B illustrate an exemplary mold 210 that can be used to form the implant 10. The mold 210 includes a mold cavity 210c that has an internal shape that corresponds to the curvature of the implant 10 with defined radii such as discussed with respect to embodiments shown in FIGS. 1, 4, and 10A-10C, for example. The mold 210 can be configured as an injection molding mold 210. The mold 210 can be a single compartment mold or a multiple compartment mold block with discrete mold cavities for fabricating a plurality of discrete implants concurrently. The mold and mold materials may be sterile or aseptic or the mold can be sterilized after it is fabricated.

FIG. 17 illustrates an example of a fabrication system/method 200 that can be used to form a porous implant 10. In this embodiments, a mold material 205 (e.g., silicone) can be mixed with a soluble porogen 208, which is molded together in the mold 210 and integrated into the molded body in a pre-form body 10a. The mold material and porogen can be premixed and introduced into the mold cavity or can be mixed while or after introduced into the mold cavity. The term “porogen” refers to a material that is able to be removed from the primary moldable material after molding in sufficient amounts to create apertures, pores or channels. The pre-form body 10a is then rinsed, sprayed, dipped, chemically reacted or otherwise processed to remove or dissolve away the porogen 208 to thereby form a porous molded body 10b. This can form a sponge-like body with interconnecting pores 40p. The porogen can comprise a salt-leeching material or other suitable material.

FIG. 18 illustrates a different example of a fabrication system/method 250. Here, the system 250 includes a laser 260 with a laser power source 265 and head 270 that is configured to form pores 40p in a molded body 10a, typically as vertical channels 40ch that extend through the implant 10. The pore channels 40ch can be substantially parallel and may have diameters as described above, e.g., between about typically between about 125 mm and 250 mm.

The methods/systems for forming the porosity described herein are by way of example only and non-limiting to the implants contemplated by the present invention. For example, the mold cavity 210c can include disposable or permanent inserts that extend in the mold to provide the apertures, pores and/or channels 40a, 40p, 40ch (not shown).

The implants 10 contemplated by embodiments of the invention can have a much smaller area than conventional slings and can optionally be implanted through a single transvaginal incision in a less invasive manner than current incontinence slings. Once fitted under the urethra, the implant 10 will typically not need to be adjusted. There is no tension on the implant itself, so the procedure is much easier to learn and reproduce consistently. The implants do not require (and can be devoid of) barbed fixation tips that may otherwise present a bleeding risk. Unlike the sling, the implants 10 do not restrict the urethra. The implants 10 can inhibit, reduce or prevent urinary leakage by dispersing or distributing force to inhibit or prevent a sudden expansion associated with stress incontinence. The surfaces of the implant can be smooth and minimize the risk of tissue erosion or organ perforation. Thus, embodiments of the present invention provide urogynecologic implants that reduce, inhibit or prevent stress incontinence by reducing the displacement of the urethra into the pelvic floor during impulses of pressure.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

That which is claimed:

1. A stress incontinence implant, comprising:
a rigid or semi-rigid urogynecologic implant comprising a first primary surface with a curved medial portion and a second primary surface underlying the first primary surface.

2. The implant of claim 1, wherein the implant comprises a monolithic solid polymer body.

3. The implant of claim 1, wherein the implant comprises a hollow interior compartment.

4. The implant of claim 1, wherein the implant has a width dimension and a length dimension, with the width dimension being greater than the length dimension.

5. The implant of claim 4, wherein the width dimensions is about 50% greater than the length dimension.

6. The implant of claim 1, wherein the second primary surface is curved and has a radius of curvature that is greater than a radius of curvature of the first primary surface medial portion.

7. The implant of claim 1, wherein the first primary surface medial portion merges into outer ramped end portions and the second primary surface has an arc configuration with opposing ends, and wherein a respective ramped end portion meets a respective arc end portion to define a respective short end edge of the implant.

8. The implant of claim 7, wherein the short end edges reside closer to the first primary surface than the second primary surface.

9. The implant of claim 1, wherein the implant is configured to reside between an anterior vaginal wall and an outer surface of a urethra.

10. The implant of claim 9, wherein the implant is held in position without mechanical fixation between the vaginal wall and the urethra.

11. The implant of claim 1, wherein the first primary surface medial portion merges into outer ramped end portions that incline down toward second primary surface, the implant further comprising mesh extending out from the respective ramped end portions.

12. The implant of claim 1, wherein the implant is sized and configured to surround only about 180 degrees or less of a female urethra and has a maximum thickness that is between about 1 mm to about 5 mm.

13. The implant of claim 1, wherein the implant has a width that is between about 15-30 mm and a length that is between about 7-10 mm and a maximum thickness that is between about 1 mm and 5 mm.

14. A method of treating urinary incontinence, comprising:
distributing forces from a first side of a three-dimensional shaped rigid or semi-rigid implant facing a urethra and residing between a urethra and anterior vagina wall of a female patient to a longer second side of the implant adjacent an anterior vaginal wall in response to impulses of abdominal pressure stress to thereby inhibit urinary incontinence.

15. A method of fabricating a urinary incontinence implant, comprising:
forming a rigid or semi-rigid three dimensional implant body having a three-dimensional shape including a first radius of curvature associated with a medial portion of a first primary surface and a second radius of curvature associated with an arc of an underlying second primary surface.
